(19) United States
(12) Patent Application Publication
(10) Pub. No.: US 2007/0287888 A1
(43) Pub. Date: Dec. 13, 2007

(54) INTEGRATED LARYNGOSCOPE AND SUCTION DEVICE

(75) Inventors: Nathan Lovell, Encinitas, CA (US); Scott Perry, San Diego, CA (US); Jessica Nelson, San Francisco, CA (US); Emily Olsen, Los Angeles, CA (US); Ryan Jaques, San Jose, CA (US)

Correspondence Address: KNOBBE MARTENS OLSON & BEAR LLP, 2040 MAIN STREET, FOURTEENTH FLOOR, IRVINE, CA, 92614, US

(73) Assignee: DP Medical, Encinitas, CA (US)

(21) Appl. No.: 11/675,602

(22) Filed: Feb. 15, 2007

Related U.S. Application Data
(60) Provisional application No. 60/804,312, filed on Jun. 9, 2006.

(51) Int. Cl. A61B 1/267

(52) U.S. Cl. 600/187

(57) ABSTRACT

A suction unit is placed inside the handle of a laryngoscope to allow portable suction to be utilized during intubation without requiring multiple devices. The suction tip can be attached to the distal end of the laryngoscope blade. Replacing the light on a laryngoscope with an LED allows for a small watch-sized battery to be used instead of current large batteries taking up the handle space. This opens up the handle space to make room for the suction unit. Evacuated fluids and discharge exit into a tube and into a portable receptacle.

### RELATED APPLICATIONS

This application claims priority to Provisional Application No. 60/804,312, entitled INTEGRATED LARYNGOSCOPE, filed Jun. 9, 2006. The subject matter of the aforementioned application is hereby incorporated herein by reference in its entirety.

### BACKGROUND OF THE INVENTION

1. Field of the Invention

This invention relates to the field of laryngoscopes and portable suction generating devices.

2. Description of the Related Art

Endotracheal intubation is a common medical procedure wherein a hollow tube is inserted down the throat, through the vocal chords and into the lungs to provide an open airway for ventilation. To perform this procedure orally, the intubator must be able to visually see the vocal chords in order to properly insert the endotracheal tube (ETT). If, for any reason, the vocal chords cannot be seen, the procedure cannot continue.

A device called a laryngoscope is used to move the tongue and lift the epiglottis to visualize the vocal chords. A complication that can occur is a fluid such as blood, vomit, or phlegm obscuring the view of the vocal chords. In this situation, a method of suction must be employed in order to clear out the airway to perform the procedure. Should the suction procedure take too long, or fail entirely, the patient is at risk for brain damage and death.

There are several types of suction methods available. Ambulances and fire trucks carry manual suction units, where the rescuer physically uses his or her hand to make the unit generate suction. This requires the removal of the laryngoscope and requires a third hand. Many of these devices are also blind; the rescuer cannot see if they are suctioning the obscuring fluids or are instead suctioning the wrong area. This also takes significant extra time to complete the intubation procedure, and the patient is not breathing. It is often times impractical to have a second rescuer supply the third hand required for the intubation and suction. In these cases, the intubator must put down either the ETT or the laryngoscope to operate the suction unit, thereby losing even more time.

Another suction method is using a mechanical suction unit with a catheter. These catheters are attached to an external suction unit, usually mounted on a wall or on a cart and require power. They are not usually blind, but do require a third hand to operate. These mechanical suction units also have tubing and tend to take up significant space.

Certain laryngoscopes can be attached to the mechanical suction catheters. This provides the advantage of removing the necessity of the third hand, and allows the intubator to insert the ETT immediately after the vocal chords can be visualized. Hospitals tend to be the only ones carrying these devices as it is impractical for use in the field, and few ambulances carry mechanical suction units. This also still requires the mechanical suction unit and tubing which impair motion and take up space.

To avoid using suction in these situations, a technique using a guidewire can be used. For this procedure, a stiff wire is inserted into the trachea with a needle, and is then fed through the throat up out the oral or nasal cavity. This wire is then used as a visual guide to place the ETT in the trachea. This is a very difficult procedure and comes with many risks. Because it is so invasive, higher levels of training are required to use this technique. It opens the patient up to infection and bleeding in the trachea which can lead to severe consequences. Even though the guide wire penetrates the obstructing fluid, if the fluid is abundant it can still obstruct the view, minimizing the usefulness of this procedure.

Further details regarding structures, devices, and methods that may be incorporated with the embodiments described below are found in the following patents, the entire disclosures of which are incorporated herein by reference: U.S. Pat. Nos. 6,569,089, 6,248,061, 7,052,456, and RE37,861.

### SUMMARY OF THE INVENTION

Embodiments of the invention herein described will help overcome the difficulties posed by obscuring fluids during endotracheal intubation. In one embodiment, a laryngoscope with a suction unit internal to the handle is provided, allowing for maximum portability while removing the need for a third hand for suction. This also allows suction to be used during the procedure such that the intubator can insert the ETT the moment the vocal chords become visible. This increases the success rate of intubations and decreases the time required when suction is necessary.

Advantageously, in one embodiment, a suction unit inside the handle of a laryngoscope allows portable suction to be utilized during intubation without requiring multiple devices. The suction tip can be attached to the distal end of the laryngoscope blade. The light on a laryngoscope may be replaced with an LED, allowing for a small watch-sized battery to be used instead of current large batteries taking up the handle space. This opens up the handle space to make room for the suction unit. Evacuated fluids and discharge exit into a tube and into a portable receptacle.

One embodiment is a laryngoscope comprising a handle and a suction unit, where the suction unit is contained within the handle. Another embodiment is a laryngoscope comprising a handle, a blade, first and second suction tubes, and a one-way valve. The blade has a proximal and distal end and is operatively connected to the handle. The first suction tube extends along the blade and is for suctioning fluids. The second suction tube is at least partially contained within the handle and is in flow communication with the first suction tube. The one-way valve is disposed between the first and second suction tubes and is in flow communication with both tubes.

Still another embodiment is a method of intubating a patient. The method comprises providing a laryngoscope having a blade, a handle, a suction unit contained within the handle, and a suction tube coupled to the blade and in flow communication with the suction unit. The method also comprises inserting the blade into the patient's throat and activating the suction unit without removing the blade from the patient's throat.

A further embodiment is a cartridge for use in a laryngoscope. The cartridge comprises a container containing a compressed gas, a valve configured to regulate the flow of the compressed gas, and a pressure gradient generator configured to accelerate the flow of the compressed gas, whereby at least a partial vacuum may be created which is suitable for suctionably withdrawing bodily fluids.

Yet another embodiment is a portable suction generating device comprising a container containing a compressed gas, a valve in flow communication with the container for regulating flow of the compressed gas, and a pressure gradient generator in flow communication with the valve and a tube. Opening the valve causes the compressed gas to flow through the pressure gradient generator and past the first end of the tube such that suction is generated at the second end of the tube.

Still another embodiment is a method of arming a portable suction device, the device comprising a container containing a compressed gas, a valve in flow communication with the container for regulating flow of the compressed gas, and a pressure gradient generator in flow communication with the valve, the valve comprising a pin configured to puncture the container. The method comprises pressing the container against the pin without rotating the container.

### BRIEF DESCRIPTION OF DRAWINGS

FIG. 1 is a left perspective view of one embodiment of the invention.

FIG. 2 is an exploded perspective view of the embodiment of FIG. 1.

FIG. 3 is a left side elevation of the handle assembly of the embodiment of FIG. 1.

FIG. 4 is a right side elevation of the handle assembly of the embodiment of FIG. 1.

FIG. 5 is a section view of the front half of the handle assembly taken along line 5-5 of FIG. 3.

FIG. 6 is a section view of the back half of the handle assembly taken along line 6-6 of FIG. 4.

FIG. 7 is an exploded view of the cartridge assembly shown in FIG. 2.

FIG. 8 is a section view of the handle head assembly shown taken along line 8-8 in FIG. 2.

FIG. 9 is an exploded view of the handle head assembly shown in FIG. 2.

FIG. 10 is a section view of the canister arming/activation mechanism shown in FIG. 5.

FIG. 11 is an exploded view of the canister arming/activation mechanism of FIG. 10.

FIG. 12 is an exploded view of the handle assembly of another embodiment of the invention.

FIG. 13 is a perspective view of the cartridge assembly of the embodiment shown in FIG. 12.

FIG. 14 is an exploded view of the cartridge assembly shown in FIG. 13.

FIG. 15 is an illustration showing the use of one embodiment of the invention in a patient to remove vomit or blood which is blocking the intubator's view of the vocal cords.

FIG. 16 is an illustration showing a successful intubation of a patient using one embodiment of the invention.

### DETAILED DESCRIPTION OF THE PREFERRED EMBODIMENTS

One embodiment of the invention is a laryngoscope with internal suction capability. All suction generating components may be located inside the handle. The laryngoscope includes a blade, and may have a tube that runs the length of the blade to the distal end, allowing a user to suction fluids without changing devices or requiring external suction units. The entire suction unit may be housed in the handle.

Embodiments with the suction unit inside the handle have particular advantages, such as allowing a user the ability to operate both the laryngoscope and the suction unit with the same hand, without removing the laryngoscope blade, even in an environment where no external suction generating unit is easily accessible. By housing the suction unit within the handle, embodiments of the invention advantageously avoid extra tubes and extra bulk near a patient's mouth, improving a user's ability to properly position the laryngoscope during an intubation procedure. Additionally, embodiments which include a suction generating unit housed in a disposable cartridge assembly desirably allow for easy and fast cleaning of a device between uses.

The laryngoscope preferably uses an LED as the light source, and includes a voltage source which is also housed in the handle. This voltage source may be a small battery of cylindrical shape, generally the size of a watch battery. The battery may be secured in the handle in a location that does not disrupt use of suction.

A suction catheter runs down one side, such as the left side of the laryngoscope blade. The suction catheter may also be a part of the blade, and be a manufactured tube internal to the blade. Suction may be generated by mechanical, manual, or other means. A switch, button, or trigger activates the suction. Should the user not use the switch, it is perpetually in the OFF position and suction does not occur. The switch may be electrical or mechanical, and may be located anywhere on the handle.

The discharge exits via a tube mounted on the handle such that it does not obstruct the user's view. Discharge then flows along this tube into a receptacle at the distal end of the tubing where it is stored for removal.

Suction may be generated by a device operating off of Bernoulli's Principle, wherein a fluid passes over an open tube at a high velocity in order to create a low pressure, thereby generating suction. This fluid may be stored in a compressed gas canister in the handle along with the Bernoulli device. A valve, controlled by a switch, prevents gas from being discharged without direct intervention by the user, and is perpetually in the OFF position. The compressed gas canister is connected to the valve. A tube takes the flow from there to the Bernoulli device. Suction is generated in this device and there is a pressure gradient between the suction tube and the Bernoulli device. In the handle, on the suction catheter there is a one way valve, oriented such that fluids may only flow towards the Bernoulli device. Fluid then flows out of the Bernoulli device and into an external tube attached to a discharge receptacle.

The handle and all internal parts may be disposable. The suction catheter may be a part of this handle. The handle may also be non-disposable but the internal parts all interconnected into a single disposable cartridge. The suction catheter may be a part of this disposable cartridge.

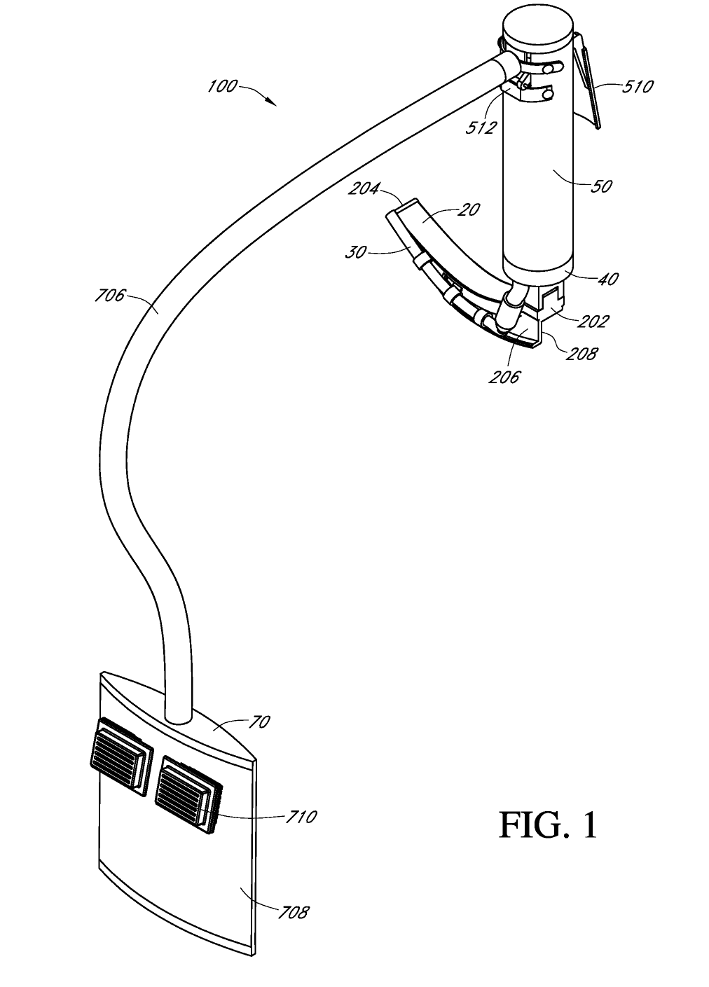

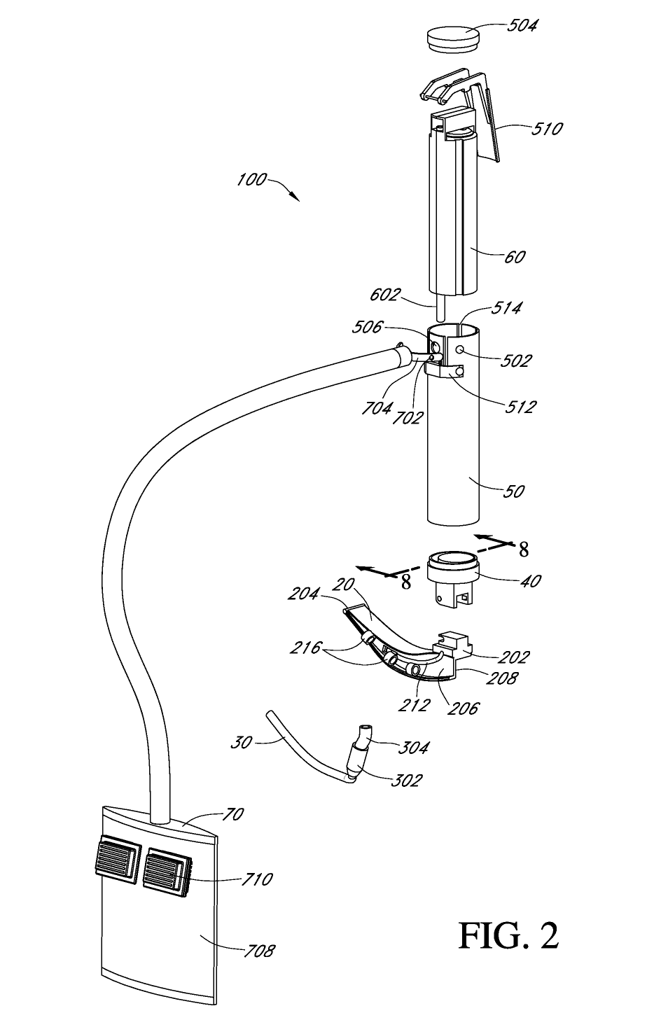

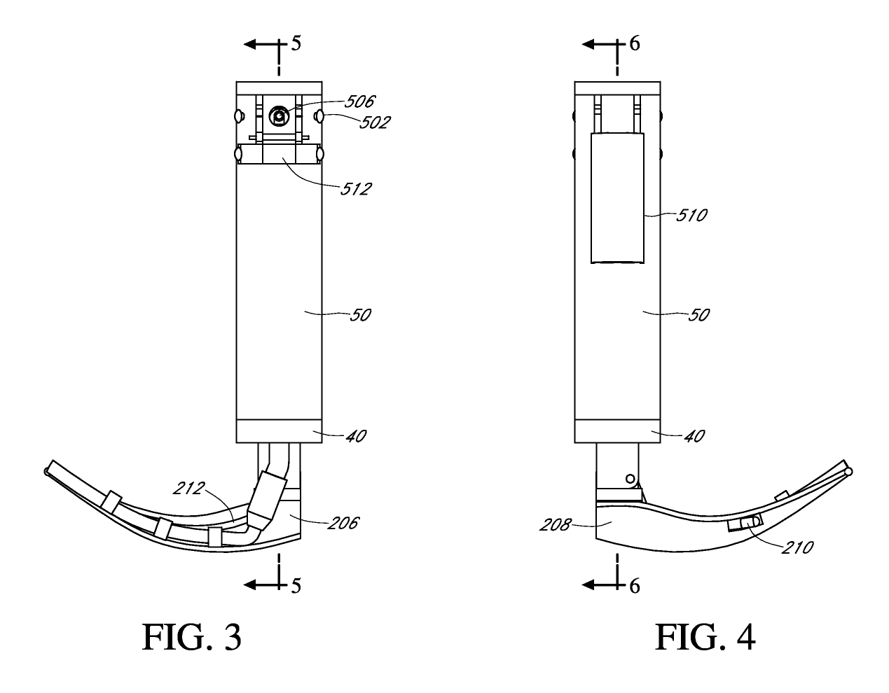

FIGS. 1-6 provide an example of a portable integrated laryngoscope and suction device 100. As shown in FIG. 1, the device 100 generally includes a blade 20, an external suction tube 30, a handle head 40, and a handle 50. As can best be seen in FIG. 2, the device 100 also includes a cartridge assembly 60 which fits inside the handle 50. The device may also include a collection assembly 70.

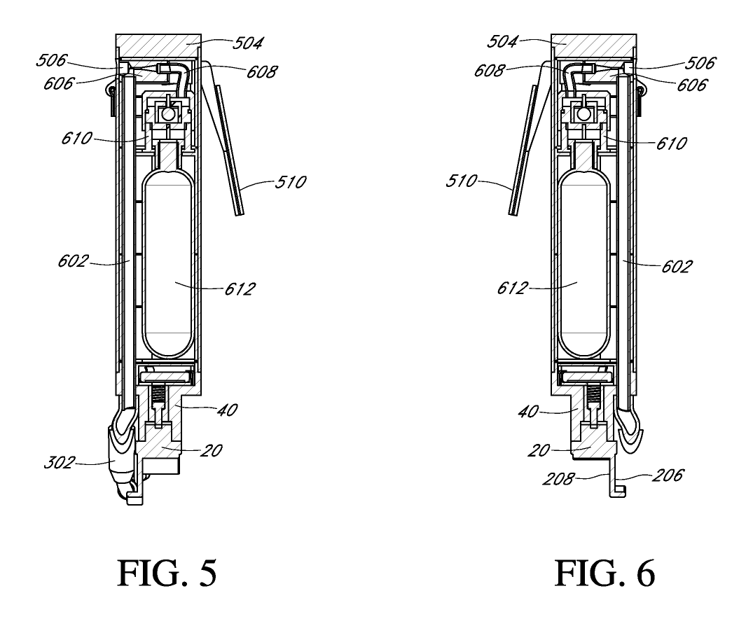

Referring now to FIG. 1, the blade 20 may have a proximal end 202 and a distal end 204. The width of the blade 20 may taper from the proximal end 202 toward the distal end 204, and the blade 20 may be either curved or straight along its length. The blade 20 may have a tongue depressor side 206, for example the left side (as viewed by a patient), which is configured to push the patient's tongue out of the way as the device 100 is inserted. The blade may also have an ETT guide side 208, for example the right side, which is configured to receive an ETT and guide it down the length of the blade 20. The blade 20 may be formed from, for example, stainless steel or coated steel, and may have a generally Z-shaped (as shown in FIGS. 5 and 6) or generally C-shaped cross section.

As best seen in FIG. 4, the ETT guide side 208 may be provided with a light source 210, attached for example near the distal end of the blade 20, which is configured to illuminate a patient's throat when the device 100 is in use. The light source 210 may be connected to a wire 212 (shown in FIG. 2) via a threaded receptacle located in a hole passing through the blade 20. The light source 210 may, for example, be an LED. The wire 212 may be attached to the blade 20 along the tongue depressor side 206 and terminate at a contact near the proximal end 202 of the blade 20.

Referring now to FIG. 2, the blade 20 may be connected to the handle head 40 by a locking hinge 214 at the proximal end 202 of the blade 20. The contact may be configured to engage a battery contact on the handle head 40 when the blade is in the locked position.

The external suction tube 30 may have an open end located near the distal end 204 of the blade 20. The tube 30 may run along the tongue depressor side 206 of the blade 20, and may be secured to the blade 20 by rings 216. The external suction tube 30 may be equipped with a one-way valve 302 which is configured to prevent backflow. The one-way valve 302 may be coupled to a connection tube 304 which attaches to an internal suction tube 602, which will be described in further detail below. The external suction tube 30 may comprise any suitable tubing material, for example ¼″ internal diameter silicon tubing. Alternatively, the external suction tube 30 may comprise an open channel formed in the blade 20 itself The connection tube 304 may comprise, for example, ⅛″ internal diameter flexible rubber or PVC tubing.

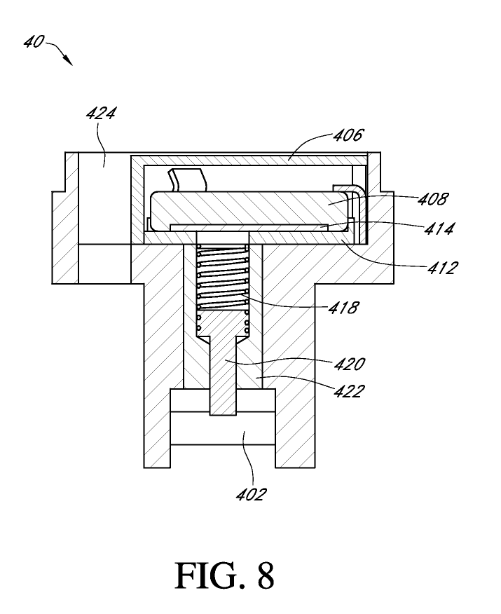

With continued reference to FIG. 2, the handle head 40 is attached to the bottom of the handle 50 via threads. The handle head 40 connects the blade 20 to the handle 50 via fixed pin 402 (shown in FIG. 8).

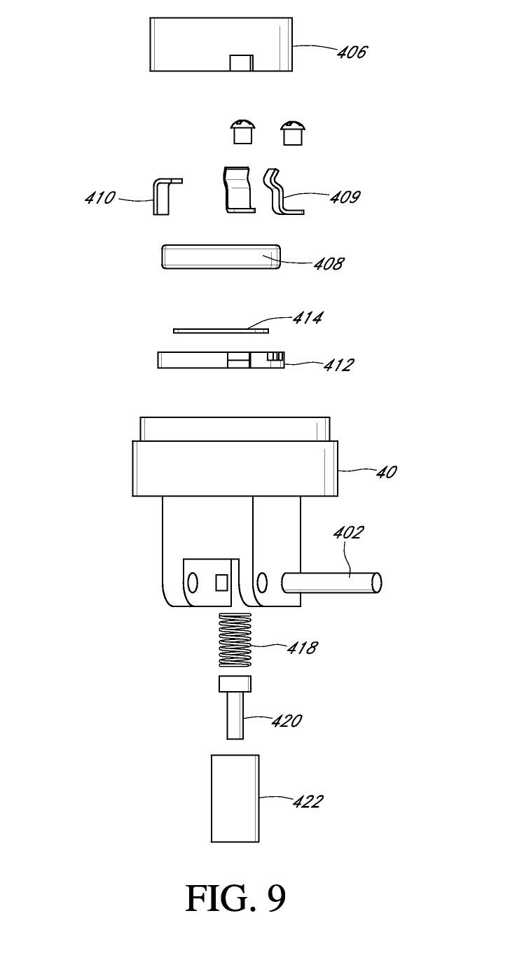

As shown in detail in FIGS. 8 and 9, the handle head 40 may house electronics to power the light 210 on the blade 20. The handle head 40 may contain a battery case 406, a battery 408, grounding hooks 409, grounding clips 410, a battery platform 412, a platform contact 414, grounding screws 416, a conductive spring 418, a conductive pin 420, and transfer casing 422. The conductive pin 420 may be configured to contact the blade 20 to transfer electrical power to the light 210 on the blade 20. These electronics are well known in the art and thus will not be described in further detail.

The handle head 40 may also include an opening 424 which is configured to receive internal suction tube 602 (which will be described in further detail below) from the cartridge assembly 70.

Referring back to FIG. 2, the handle 50 may house the cartridge assembly 60. The handle 50 may include two external attachment pins 502 which mate with holes 702 in a collection seal 704 to secure the collection seal 704 to the handle 50. The handle may further include internal nuts (not shown) located on the inside of the handle 50 opposite the attachment pins 502, configured to hold the attachment pins 502 in place and to guide the cartridge assembly 60 into the handle 50 in an appropriate position. A handle cap 504, configured to secure the cartridge assembly 60 inside the handle 50, may mate with the top end of the handle 50 via threads.

The handle 50 may further include an exit hole 506 which is configured to provide an exit route for fluids being suctioned through the internal suction tube 602 to the collection system 70. The handle 50 may also include a trigger 510 which is configured to control suction in the device 100. The trigger 510 may be configured to engage an actuator 644 (described in further detail below) when the trigger 510 is depressed. The trigger 510 may be located in any appropriate position along the blade, and may be an electrical or mechanical trigger. The trigger 510 may be formed from any suitable material, for example, machined aluminum. The trigger 510 may be pivotably connected via a hingepin to a trigger hinge 512 attached to the opposite side of the handle 50. The trigger hinge 512 may be formed from, for example, steel or another sheetmetal, and may be attached to the handle 50 in any suitable manner. The handle 50 may include slots 514 to allow the trigger 510 to pivot freely around the trigger hinge 512 when the handle cap 504 is removed.

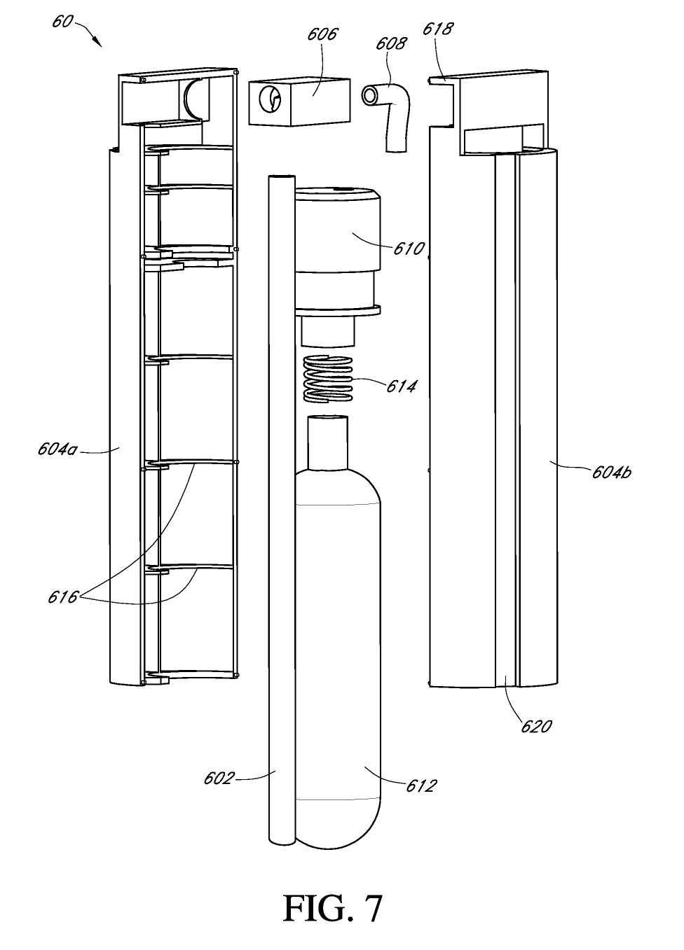

With reference now to FIG. 7, the cartridge assembly 60 may include a housing comprising a front mold 604(a) and a back mold 604(b). The cartridge assembly may also house a suction unit or source of suction. One embodiment includes a suction unit comprising the internal suction tube 602, a pressure gradient generator 606, a flow feed tube 608, a canister arming/activation mechanism 610, an energy source comprising a compressed gas canister 612, and a canister spring 614.

As described above, the internal suction tube 602 may fit through the opening 424 in the handle head 40, and may be connected to the connection tube 304 in the suction tube assembly 30. The internal suction tube 602 may lead from the connection tube 304 up toward the top of the cartridge assembly 60, where it may connect with an opening in the pressure gradient generator 606. The flow feed tube 608 may be connected to the canister arming/activation mechanism 610 and may be configured to carry high velocity gas released from the gas canister 612 to the pressure gradient generator 606. As shown in FIGS. 5-7, the pressure gradient generator 606 may be configured to accelerate the air released from the gas canister 612 over the top of the internal suction tube 602 and thereby generate a pressure gradient in the suction tube 602.

The canister spring 614 may be positioned between the gas canister 612 and the canister arming/activation mechanism 610, and may be configured to mitigate any jarring motion that may occur in an ambulance or during shipping from an accidental puncturing of the gas canister 612.

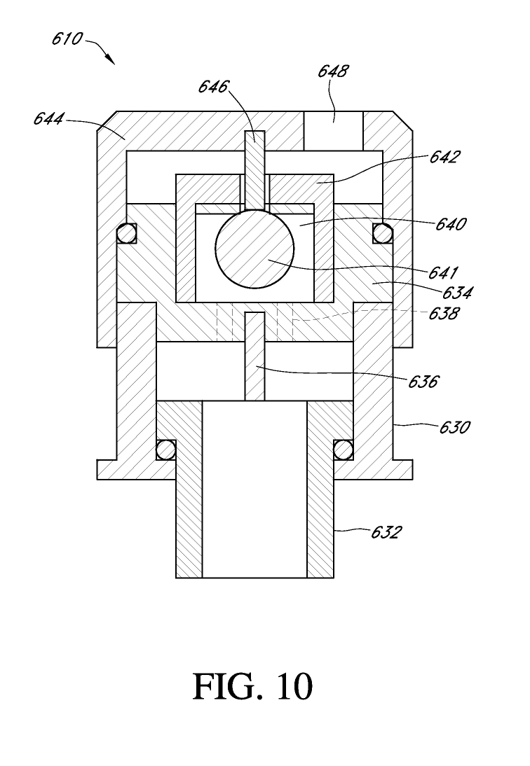

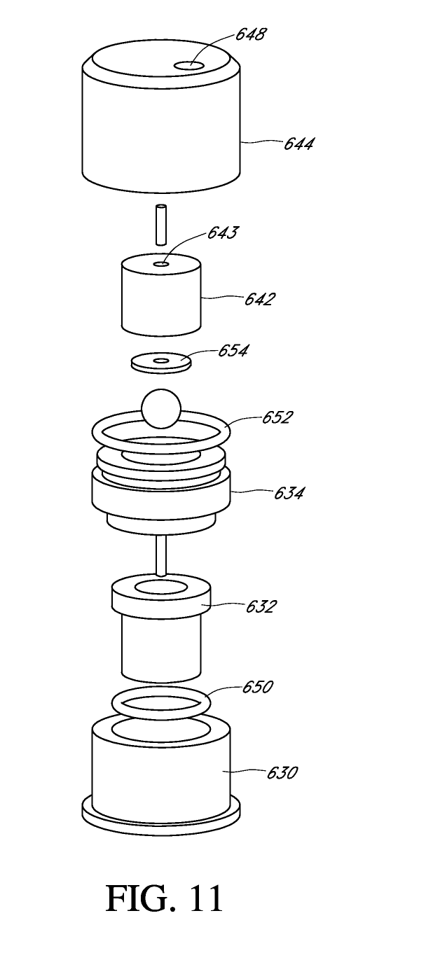

With reference now to FIGS. 10 and 11, the canister arming/activation mechanism 610 may include an arming portion 630 located above the gas canister 612. The arming portion 630 may hold a canister collar 632 and a puncture portion 634. The canister collar 632 may be threaded to receive the neck of the gas canister 612. The puncture portion 634 may include a puncture pin 636 which is configured to puncture the gas canister 612 when a user presses the gas canister 612 into the cartridge assembly 60, thereby releasing pressurized gas into the mechanism 610 and arming the cartridge assembly 60. The puncture portion 634 may further include holes 638 which are configured to allow released gas to flow into a chamber 640, located above the puncture portion 634 and within a bearing casing 642. The arming portion 630 and the bearing casing 642 may be attached to the puncture portion 634 via threads.

The canister arming/activation mechanism 610 may further include an actuator 644, located at the top of the arming/activation mechanism 610 generally above the arming portion 630. The actuator 644 may include a pin 646 which is configured to contact a free-floating ball bearing 641 through a hole 643 in the top of the bearing casing 642. The ball bearing 641 may be located within the chamber 640, and may be configured to block leakage of pressurized gas from the chamber 640 through the hole 643 after the gas canister 612 has been initially punctured (armed). When the activation portion 644 is depressed, the pin 646 may be configured to depress the ball bearing 641, allowing gas to flow through the hole 643 and out of the mechanism 610 via a hole 648 at the top of the activation portion 644, thereby activating the mechanism 610.

As can best be seen in FIG. 11, the mechanism 610 may be equipped with an o-ring 650 disposed between the arming portion 630 and the canister collar 632, to seal in released gas after the gas canister 612 has been armed. The mechanism 610 may further include an o-ring 652 disposed between the puncture portion 634 and the actuator 644, to prevent leakage once the mechanism 610 is activated. The mechanism 610 may also include a bearing gasket 654, configured to help the ball bearing 641 seal the hole 643 when the mechanism 610 is armed but not activated.

Referring again to FIG. 7, the cartridge molds 604 may mate together via pins, and may be secured together with an adhesive such as glue. The molds 604 may include internal ribs 616 which are configured to secure the gas canister 612 and the canister arming/activation mechanism 610 within the cartridge assembly 60. When mated together, the molds 604 may define a channel 618 which is configured to closely receive and align the exit port end of the pressure gradient generator 606 with the exit hole 506 in the handle 50. The molds 604 may also be configured to allow the trigger 510 to pivot around the trigger hinge 512 and depress the trigger valve depressor 644 when the device 100 is assembled. Each mold 604 may also include an open channel 620 which is configured to receive one of the cartridge guide nuts on the inside of the handle 50 and guide the cartridge assembly into the handle 50 in an appropriate position. When mated together, the molds 604 may further define an opening at the bottom of the cartridge assembly 60, through which a user may press on the end of the gas canister 612 and thereby arm the canister assembly 60. For ease of cleaning and for re-usability of the device 100, the entire cartridge assembly 60 may be disposable.

It will be apparent to one skilled in the art that other embodiments may include different types of suction generating units from that described above. It will also be apparent that other configurations are possible as well, as shown in FIGS. 1 and 2A of Provisional Application No. 60/804,312, which is incorporated by reference in its entirety.

Referring again to FIGS. 1 and 2, the collection assembly 70 may include a collection hose 706 which terminates inside a collection bag 708. The end of the collection hose 706 may be provided with a condensing tube (not shown) configured to facilitate condensation of atomized fluids. The collection bag 708 may be provided with air filters 710 which are located near the top of the bag 708 and which are configured to prevent atomized fluids from being expelled into the air.

The collection seal 704 may be formed from any material capable of providing an appropriate seal between the cartridge assembly 60 and the collection hose 706, for example, neoprene rubber. The collection hose 706 may consist of any appropriate material, for example kink-resistant EVA hosing. The condensing tube 710 may be formed from any suitable tubing material, for example, plastic. The collection bag 708 may also be formed from any appropriate material, such as a heavy flexible plastic.

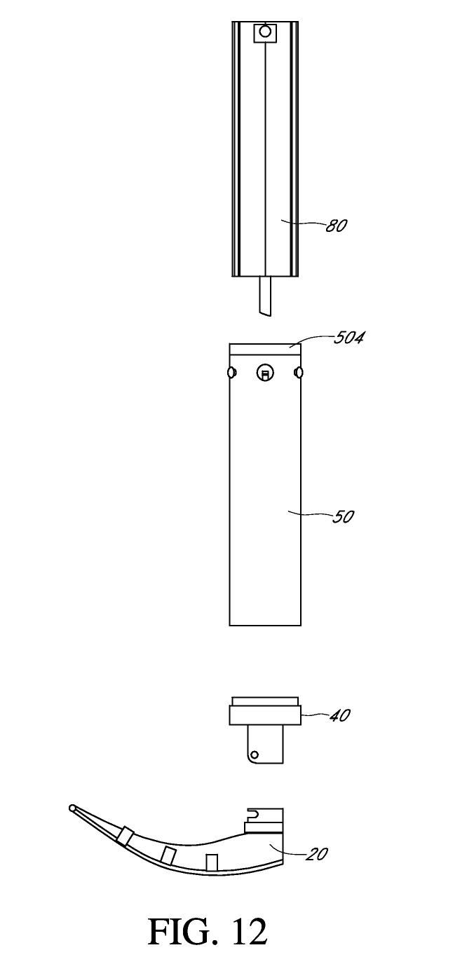

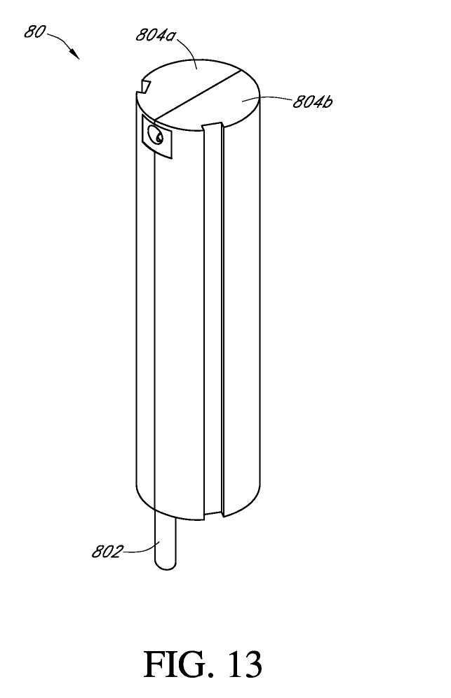

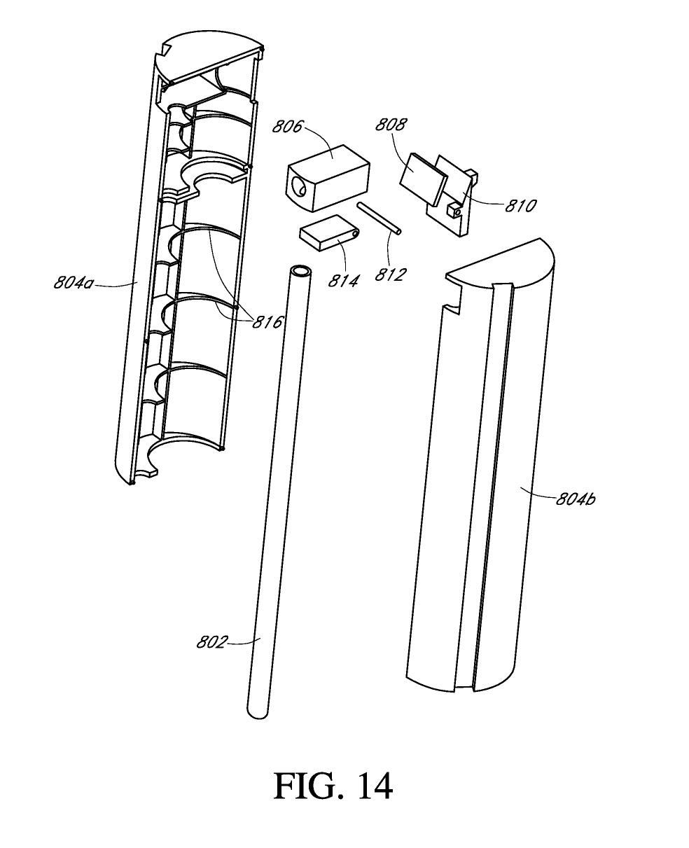

With reference now to FIG. 12, an alternative embodiment of the invention may include a cartridge assembly 80 which attaches to an external suction generator (not shown). As best shown in FIGS. 13 and 14, the cartridge assembly 80 may include an internal suction tube 802, a front mold 804(a), a back mold 804(b), a pressure T-joint 806, a breaker seal 808, and a breaker lever 810. The cartridge molds 804 may include internal ribs 816 to secure the pressure T joint 806 and the internal suction tube 802 in place. The pressure T-joint 806 may be connected to the top of the internal suction tube 802 and may provide multiple fluid channels for suction and suctioned fluids. The breaker seal 808 may be attached to the breaker lever 810 via a hingepin 812 and a hinge 814. The breaker lever 810 may be provided with a spring (not shown) which is configured to hold the breaker lever 810 in a default position. The breaker seal 808 may be configured to remain open and prevent suction when the breaker lever 810 is in the default position, and engage suction through the pressure T joint 806 when the breaker lever 810 is depressed.

To assemble the device as described in FIGS. 1-11, a user activates the cartridge assembly 60 by pushing on the gas canister 612 through the hole in the bottom of the cartridge assembly 60. This causes the puncture pin 636 to puncture the canister 612, thereby arming the cartridge assembly 60. The user then removes the handle cap 504 from the top of the handle, and flips the trigger 510 to the outside of the handle 50. The user then drops the cartridge 60 into the handle 50, aligning the open channels 620 with the pins on the inside of the handle 50. This ensures proper placement of the cartridge 60 in the handle 50. Then the user secures the handle cap 504 to the handle 50. Alternatively, the cartridge assembly 60 may be configured to allow activation of the assembly 60 while it is inside the handle 50.

Next, the user inserts the external suction tube 30 through the rings 216 on a properly sized blade 20. The user then hooks the blade 20 onto the handle head 40 and connects the connection tube 304 with the bottom end of the internal suction tube 602. The user then secures the external collection hose 706 to the exit hole 506 in the handle 50, attaching the collection seal 704 to the handle 50 at attachment points 502. Once the device 100 is armed and assembled, the user may engage suction in the device 100 (that is, the user may activate the device 100) by depressing the trigger 510. The user may release suction by releasing the trigger 510.

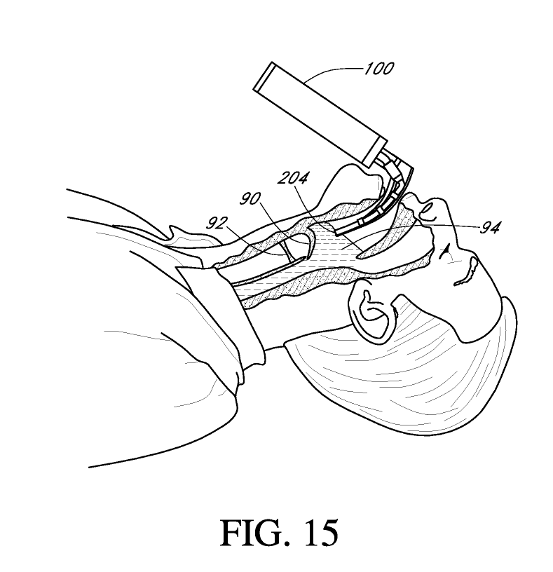

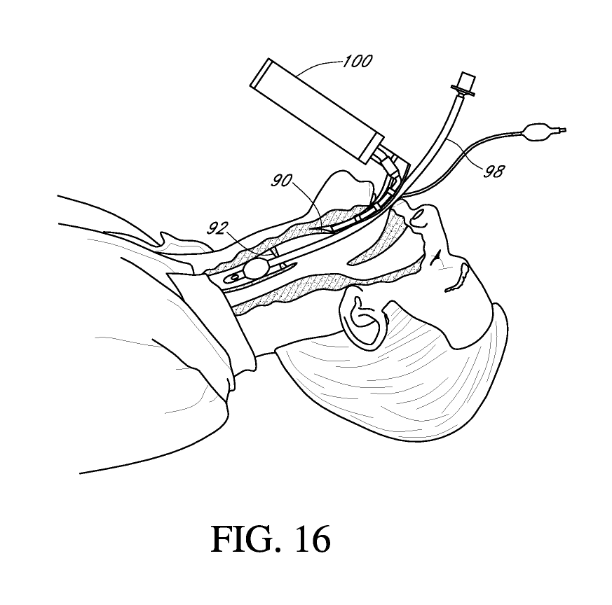

A method of intubating a patient is also disclosed. FIG. 15 is an illustration of a patient requiring intubation but having the intubator's view of the epiglottis 90 and vocal cords 92 occluded by bodily fluids 94. As shown in the figure, a user may insert the device 100 into the patient's throat, determine whether the view of the epiglottis and vocal cords is occluded, and, if necessary, activate suction within the device 100 by depressing the trigger 510 (not visible in the figure). The user may adjust the location of the distal end of the blade 204 in order to optimize removal of fluids. As shown in FIG. 16, once the vocal cords 92 become visible, the user may terminate suction in the device 100 by releasing the trigger 510. The user may then immediately insert the ETT 98 along the ETT guide side 208 (opposite the side visible in the figure) of the blade 20 and through the vocal cords 92. The collection assembly 70 and the trigger hinge 512 are not depicted in FIGS. 5 and 6.

Because the suction tube 30 is preferably located on the tongue depressor side 206 of the blade 20, the presence of the suction tube does not impede insertion of the ETT. Because the suction unit is contained within the device 100, the intubator may successfully intubate a patient having occluding fluids in his throat without the intermediate step of removing the laryngoscope to insert an external suction device, thereby saving valuable seconds. Further, because the entire device 100 is portable, the method of the invention can be used to successfully intubate such a patient when an external suction generating unit is not available, for example, in a field environment.

It will be understood by those of skill in the art that numerous and various modifications can be made without departing from the spirit of the present invention. Therefore, it should be clearly understood that the forms of the invention are illustrative only and are not intended to limit the scope of the invention.

What is claimed is:

1. A laryngoscope comprising:
a handle; and
a suction unit contained within the handle.

2. The laryngoscope of claim 1, wherein the suction unit is fully contained within the handle.

3. The laryngoscope of claim 1, wherein the suction unit is partially contained within the handle.

4. The laryngoscope of claim 1, further comprising a suction tube operatively connected to the suction unit.

5. The laryngoscope of claim 4, the suction tube having an internal diameter of between about 1/32 inch and about ½ inch.

6. The laryngoscope of claim 1, further comprising a blade, the blade being removably attached to the handle.

7. The laryngoscope of claim 6, further comprising a light source disposed along the blade.

8. The laryngoscope of claim 1, further comprising a switch for activation of the suction unit.

9. The laryngoscope of claim 8, wherein the switch is a mechanical trigger positioned on the handle.

10. The laryngoscope of claim 1, further comprising a receptacle, the receptacle being operatively connected to the handle so as to capture fluids withdrawn by the suction unit.

11. The laryngoscope of claim 1, wherein the suction unit comprises:
a container containing a compressed gas;
a valve in flow communication with the container for regulating flow of the compressed gas; and
a pressure gradient generator in flow communication with the valve and in flow communication with a tube having a first and second end, wherein opening the valve causes the compressed gas to flow through the pressure gradient generator and past the first end such that suction is generated at the second end.

12. The laryngoscope of claim 11, further comprising a one-way valve in flow communication with second end of the tube.

13. The laryngoscope of claim 1, further comprising:
a blade operatively connected to the handle;
a light source connected to the blade;
a tube in flow communication with the suction unit and attached to the blade; and
a switch for activation of the suction unit.

14. A laryngoscope comprising:
a handle;
a blade having a proximal end and a distal end, the blade being operatively connected to the handle;
a first suction tube extending along the blade for suctioning fluids;
a second suction tube at least partially contained within the handle and having a first and second end, the second end being in flow communication with the first suction tube; and
a one-way valve disposed between and in flow communication with the first and second suction tubes.

15. The laryngoscope of claim 14, further comprising a suction generating unit at least partially contained within the handle and operatively connected to the second suction tube.

16. The laryngoscope of claim 15, further comprising a cartridge configured to house the suction generating unit.

17. The laryngoscope of claim 15, wherein the suction generating unit comprises:
a container containing a compressed gas;
a valve configured to regulate the flow of the compressed gas; and
a pressure gradient generator configured to accelerate the flow of the compressed gas; whereby at least a partial vacuum may be created in the second suction tube which is suitable for suctionably withdrawing bodily fluids.

18. The laryngoscope of claim 16, wherein the cartridge and the suction generating unit are disposable.

19. The laryngoscope of claim 14, further comprising a light source attached to the blade.

20. The laryngoscope of claim 14, further comprising a receptacle configured to capture bodily fluids withdrawn by the suction generating unit.

21. The laryngoscope of claim 15, further comprising a switch disposed on the handle and configured to activate suction in the suction generating unit.

22. A method of intubating a patient, the method comprising:
providing a laryngoscope comprising a blade, a handle, a suction unit contained within the handle, and a suction tube coupled to the blade and in flow communication with the suction unit;
inserting the blade into the patient's throat; and
activating the suction unit without removing the blade from the patient's throat.

23. The method of claim 22, further comprising illuminating the patient's throat with a light source coupled to the blade.

24. The method of claim 22, further comprising deactivating the suction unit.

25. The method of claim 22, further comprising inserting an endotracheal tube into the patient's throat next to the blade.

26. The method of claim 22, wherein the suction unit is substantially contained within a cartridge.

27. The method of claim 22, wherein the suction unit comprises a pressure gradient generator configured to accelerate the flow of a compressed gas over an end of the suction tube; whereby at least a partial vacuum may be created in the suction tube which is suitable for suctionably withdrawing bodily fluids.

28. The method of claim 27, wherein the suction unit further comprises a valve configured to regulate the flow of the compressed gas.
